(19) United States
(12) Patent Application Publication
(10) Pub. No.: US 2024/0304333 A1
(43) Pub. Date: Sep. 12, 2024

(54) APPARATUS AND METHOD FOR PREDICTING OCCURRENCE OF MOOD EPISODE USING DIGITAL PHENOTYPE

(71) Applicant: KOREA UNIVERSITY RESEARCH AND BUSINESS FOUNDATION, Seoul (KR)

(72) Inventor: Heon Jeong LEE, Seoul (KR)

(21) Appl. No.: 18/472,832

(22) Filed: Sep. 22, 2023

(30) Foreign Application Priority Data
Mar. 6, 2023 (KR) 10-2023-0028938

(51) Int. Cl. G16H 50/30

(52) U.S. Cl. CPC G16H 50/30

(57) ABSTRACT

A method for predicting an occurrence of a mood episode using a digital phenotype according to the exemplary embodiment may include acquiring log data associated with a circadian rhythm of a target user from at least one of a user terminal of the target user and a wearable device which is attached to or worn on a body of the target user, extracting predetermined main feature information from the log data, and deducing prediction information about the occurrence of the mood episode of the target user by inputting the main feature information to a previously trained artificial-intelligence based prediction model.

### CROSS-REFERENCE TO RELATED APPLICATIONS

This application claims the priority of Korean Patent Application No. 10-2023-0028938 filed on Mar. 6, 2023, in the Korean Intellectual Property Office, the disclosure of which is incorporated herein by reference.

### BACKGROUND

### Field

The present disclosure relates to an apparatus and a method for predicting an occurrence of a mood episode using a digital phenotype. For example, the present disclosure relates to a technique of predicting the imminent recurrence of mood episodes using real-time digital phenotypes of a user with mood disorders, such as depression and bipolar disorder.

### Description of the Related Art

Mood disorders including a major depressive disorder (MDD), a bipolar I disorder, and a bipolar II disorder have a high recurrence rate and a mood, sleep, and vitality of a patient may be in continuously unstable state.

Therefore, for the preemptive management, prevention, and successful treatment of symptoms of mood disorders, it is important to manage the recurrence of the mood episode and for better prognosis, a new approach is necessary to evaluate, analyze, and manage the daily condition of the patient together with the pharmacological and psychotherapeutic treatments of the related art.

Further, as the association between the mental illness and sleep disorder has been known, it has been widely known that the circadian rhythm imbalance is closely related to the mental disorders. While insomnia or hypersomnia is a major symptom of the depressive episode, the reduction in the necessity of the sleep is a feature of the manic episode.

As described above, the sleep-wake problems may obviously occur even during periods in which the mood disorders are relatively stable and deterioration in the sleep-wake disorder is known to accompany or precede relapse of a mood episode in the clinical environment. Therefore, in the case of the mood disorder, it is necessary to carefully monitor the sleep during the period with regard to the episode relapse.

In the meantime, the digital technology and artificial intelligence-based analysis technology which are rapidly developing in recent years change the medical field while overcoming various limitations of the existing medicine. Unlike the psychiatric care of the related art which significantly depends on the self-report and inevitably integrates the subjective judgement of an observer close to the anxious patient and recall bias, with the advent of the wearable device and the smartphone, the mental state of the patient is inferred from the daily life pattern of the patient and further, a clinician may assist to identify a patient at a risk of the imminent relapse of the mood episodes.

Therefore, the field of psychiatry is expected to obtain the big advantage through an exact phenotype and the computer modeling of the temporal elapse of the disease onset and relapse.

A related art of the present disclosure is disclosed in Korean Patent No. 10-2303172.

### SUMMARY

An object to be achieved by the present disclosure is to provide an apparatus and a method for predicting occurrence of a mood episode using a digital phenotype which deduce prediction information about the occurrence of a mood episode of a user by analyzing log data collected by a user terminal and a wearable device.

However, objects to be achieved by various embodiments of the present disclosure are not limited to the technical objects as described above and other technical objects may be present.

As a technical means to achieve the above-described technical object, a method for predicting an occurrence of a mood episode using a digital phenotype according to the exemplary embodiment of the present disclosure may include acquiring log data associated with a circadian rhythm of a target user from at least one of a user terminal of the target user and a wearable device which is attached to or worn on a body of the target user; extracting predetermined main feature information from the log data; and deducing prediction information about the occurrence of the mood episode of the target user by inputting the main feature information to a previously trained artificial-intelligence based prediction model.

Further, in the deducing of prediction information, information of an occurrence possibility of at least one of a major depressive episode, a manic episode, and a hypomanic episode of the target user may be calculated as the prediction information within a predetermined analysis period.

Further, the method for predicting an occurrence of a mood episode using a digital phenotype according to the exemplary embodiment of the present disclosure may include acquiring user information including disease information about a mood disorder type of the target user.

In the deducing of prediction information, the prediction information may be deduced using a target prediction model which is determined so as to correspond to the disease information, among a plurality of previously trained prediction models.

Further, the mood disorder type may include a major depressive disorder, a bipolar I disorder, and a bipolar II disorder.

Further, a type of the main feature information which is input to the target prediction model and a weight of the main feature information may be determined in consideration of the disease information.

Further, the main feature information may include feature information corresponding to each of a plurality of categories including heart rate information, light exposure information, sleep information, and step information of the target user.

A training method of an artificial-intelligence prediction model for predicting a relapse of a mood episode using a digital phenotype according to an exemplary embodiment of the present disclosure may include acquiring log data associated with a circadian rhythm of a plurality of users collected from at least one of a user terminal of each of the plurality of users and a wearable device which is attached to or worn on a body of each of the plurality of users; preparing answer data associated with occurrence of mood episode of each of the plurality of users; selecting main feature information associated with the mood episode occurrence, among a plurality of feature information extracted from the log data based on the log data and the answer data; and building an artificial-intelligence based prediction model which outputs prediction information about mood episode occurrence of the target user using the main feature information, when the log data of the target user is input.

Further, the log data and the answer data may be classified into a plurality of groups depending on a mood disorder type of each of the plurality of users.

Further, in the building of the prediction model, a plurality of prediction models corresponding to the mood disorder type may be trained using the classified log data and the answer data.

Further, an apparatus for predicting an occurrence of a mood episode using a digital phenotype according to the exemplary embodiment of the present disclosure may include a log collecting unit configured to acquire log data associated with a circadian rhythm of a target user from at least one of a user terminal of the target user and a wearable device which is attached to or worn on a body of the target user; a feature extracting unit configured to extract predetermined main feature information from the log data; and an analyzing unit configured to deduce prediction information about the relapse of the mood episode of the target user by inputting the main feature information to a previously trained artificial-intelligence based prediction model.

Further, the analyzing unit may calculate information of an occurrence possibility of at least one of a major depressive episode, a manic episode, and a hypomanic episode of the target user as the prediction information within a predetermined analysis period.

Further, the log collecting unit may acquire user information including disease information about a mood disorder type of the target user.

Further, the analyzing unit may deduce the prediction information using a target prediction model which is determined so as to correspond to the disease information, among a plurality of previously trained prediction models.

In the meantime, a training apparatus of an artificial-intelligence prediction model for predicting a relapse of a mood episode using a digital phenotype according to an exemplary embodiment of the present disclosure may include a data collecting unit configured to collect log data associated with a circadian rhythm of a plurality of users collected from at least one of a user terminal of each of the plurality of users and a wearable device which is attached to or worn on a body of each of the plurality of users and prepare answer data associated with occurrence of mood episode of each of the plurality of users; a feature analyzing unit configured to select main feature information associated with the mood episode occurrence, among a plurality of feature information extracted from the log data based on the log data and the answer data; and a model training unit configured to build an artificial-intelligence based prediction model which outputs prediction information about mood episode occurrence of the target user using the main feature information, when the log data of the target user is input.

Further, the model training unit may train a plurality of prediction models corresponding to the mood disorder type using the classified log data and the answer data.

The above-described solving means are merely illustrative but should not be construed as limiting the present disclosure. In addition to the above-described embodiments, additional embodiments may be further provided in the drawings and the detailed description of the present disclosure.

According to the present disclosure described above, it is possible to provide an apparatus and a method for predicting an occurrence of a mood episode using a digital phenotype which may deduce prediction information about the occurrence of a mood episode of a user by analyzing log data collected by a user terminal and a wearable device.

However, the effect which may be achieved by the present disclosure is not limited to the above-described effects, there may be other effects.

### BRIEF DESCRIPTION OF DRAWINGS

The above and other aspects, features and other advantages of the present disclosure will be more clearly understood from the following detailed description taken in conjunction with the accompanying drawings, in which:

FIG. 1 is a schematic diagram of a digital health care system including an apparatus for predicting an occurrence of a mood episode using a digital phenotype according to an exemplary embodiment of the present disclosure;

FIGS. 2A and 2B are views for explaining a category of feature information deduced from log data collected for a user;

FIG. 3 is a view for explaining a detailed type of feature information deduced from log data collected for a user;

FIGS. 4A to 4C are graphs visualizing and illustrating main feature information corresponding to each of a plurality of prediction models for predicting an occurrence possibility of a major depressive episode, a manic episode, and a hypomanic episode, respectively, and the degree of influence of each of the main feature information on the output of the prediction model;

FIG. 5 is a table illustrating a prediction performance of each artificial intelligence-based prediction model of an apparatus for predicting an occurrence of a mood episode using a digital phenotype according to an exemplary embodiment of the present disclosure, according to a disease type;

FIG. 6 is a schematic diagram of an apparatus for predicting an occurrence of a mood episode using a digital phenotype according to an exemplary embodiment of the present disclosure;

FIG. 7 is an operation flowchart of a method for predicting an occurrence of a mood episode using a digital phenotype according to an exemplary embodiment of the present disclosure; and

FIG. 8 is an operation flowchart of a training method of an artificial intelligence-based prediction model for predicting a relapse of a mood episode using a digital phenotype according to an exemplary embodiment of the present disclosure.

### DETAILED DESCRIPTION OF THE EMBODIMENT

Hereinafter, the present disclosure will be described more fully hereinafter with reference to the accompanying drawings, in which exemplary embodiments of the present disclosure are shown. However, the present disclosure may be realized in various different forms and is not limited to the embodiments described herein. Accordingly, in order to clearly explain the present disclosure in the drawings, portions not related to the description are omitted. Like reference numerals designate like elements throughout the specification.

Throughout this specification and the claims that follow, when it is described that an element is “coupled” to another element, the element may be “directly coupled” to the other element or “electrically coupled” or “indirectly coupled” to the other element through a third element.

Through the specification of the present disclosure, when one member is located “on”, “above”, “on an upper portion”, “below”, “under”, and “on a lower portion” of the other member, the member may be adjacent to the other member or a third member may be disposed between the above two members.

In the specification of the present disclosure, unless explicitly described to the contrary, the word “comprise” and variations such as “comprises” or “comprising”, will be understood to imply the inclusion of stated elements but not the exclusion of any other elements.

The present disclosure relates to an apparatus and a method for predicting an occurrence of a mood episode using a digital phenotype. For example, the present disclosure relates to a technique of predicting the imminent recurrence of mood episodes using real-time digital phenotypes of a user with mood disorders, such as depression and bipolar disorder.

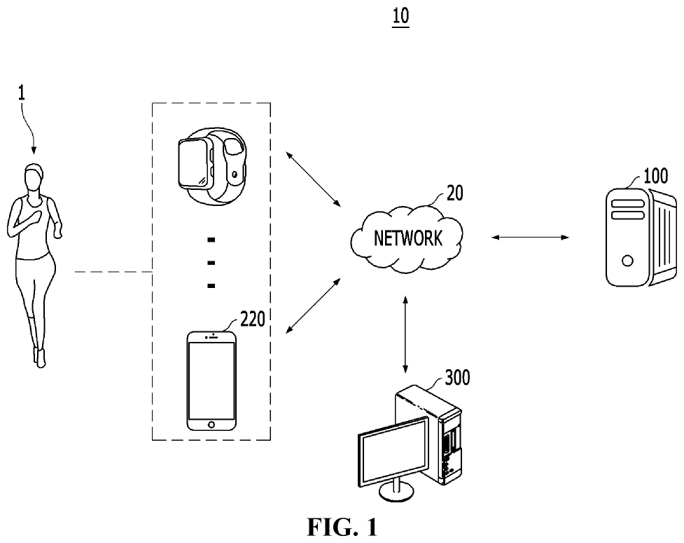

FIG. 1 is a schematic diagram of a digital health care system including an apparatus for predicting an occurrence of a mood episode using a digital phenotype according to an exemplary embodiment of the present disclosure.

Referring to FIG. 1, a digital health care system according to an exemplary embodiment of the present disclosure may include an apparatus 100 for predicting (hereinafter, referred to as a “predicting apparatus 100”) an occurrence of a mood episode using a digital phenotype according to an exemplary embodiment of the present disclosure, a wearable device 210, a user terminal 220, and a medical staff terminal 300.

The predicting apparatus 100, the wearable device 210, the user terminal 220, and the medical staff terminal 300 may communicate with each other by means of a network 20. The network 20 means a connection structure which allows information exchange between nodes such as terminals or servers. Examples of the network 20 include a 3rd generation partnership project (3GPP) network, a long term evolution (LTE) network, a 5G network, a world interoperability for microwave access (WIMAX) network, Internet, a local area network (LAN), a personal area network (PAN), a Wi-Fi network, a Bluetooth network, a satellite broadcasting network, an analog broadcasting network, and a digital multimedia broadcasting (DMB) network, but are not limited thereto. Further, the predicting apparatus 100 according to the exemplary embodiment of the present disclosure may be implemented to be loaded (installed) in at least one of the wearable device 210 and the user terminal 220.

For example, the wearable device 210 may be a smart watch or a smart band which is worn on the wrist of a user to collect biometric information of the user, but is not limited thereto. As another example, the wearable device 210 may broadly refer to a device which is worn on various positions to acquire feature information of the user 1, such as a head-worn device, a strap type device, a garment-type device, or shoe-worn/foot pods device. Further, for example, the wearable device 210 may be Fitbit, Jawbone Up, Nike+ FuelBand, Apple Watch, or Samsung Gear. Further, the wearable device 210 and the user terminal 220 may be interlinked based on the same account information for one user 1. Further, according to an implemented example of the present disclosure, it may be understood that the wearable device 210 is included in the user terminal 220 in a broad sense.

In the description of the exemplary embodiment of the present disclosure, the medical staff terminal 300 refers to a device held by a doctor of the user 1, medical staff, or guardian to acquire prediction information for the occurrence of the mood episode of the user from the predicting apparatus 100 to identify a state of the user. When the occurrence of a specific type of mood episode (for example, a major depressive episode (MDE), a manic episode (ME), or a hypomanic episode (HME)) is predicted, the medical staff terminal generates and transmits guide information including an appropriate action to be taken by the user 1 to the user terminal 220 and/or the wearable device 210.

For example, the user terminal 220 and/or the medical staff terminal 300 may include a smart phone, a smart pad, and a tablet PC, and terminals of all kinds of wireless communications such as personal communication system (PCS), global system for mobile communication (GSM), personal digital cellular (PDC), personal handy phone system (PHS), personal digital assistant (PDA), international mobile communication (IMT)-2000, code division multiple access (CDMA)-2000, W-code division multiple access (W-CDMA), and wireless broadband internet (Wibro).

In the description of the exemplary embodiment of the present disclosure, the predicting apparatus 100 may also be referred to as a “training apparatus 100” which performs the learning (training) of an artificial intelligence-based prediction model to predict a relapse of a mood episode using a digital phonotype. In other words, in the description of the exemplary embodiment of the present disclosure, the reference numeral 100 may be exchangeably used for the apparatus 100 for predicting an occurrence of a mood episode using a digital phenotype and a training apparatus 100 of an artificial intelligence-based prediction model to predict a relapse of a mood episode using a digital phenotype.

Hereinafter, the specific function and operation of the predicting apparatus 100 will be described in detail.

The predicting apparatus 100 may acquire log data related to a circadian rhythm of a target user 1 from at least one of the user terminal 220 of the target user 1 and a wearable device 210 which is attached to or worn on a body of the target user 1. According to the exemplary embodiment of the present disclosure, the log data may include physical activity related information of a user which may be acquired from the user terminal 220 and/or the wearable device 210 of the target user 1, position information, a usage record (log) of a specific application, on-off information of a screen, time stamp information, biometric signal information, and measurement data of a sensor module, but is not limited thereto.

Further, the predicting apparatus 100 may extract predetermined main feature information from the collected log data. The predicting apparatus 100 disclosed in the present disclosure with regard to this may utilize predetermined feature information deduced from the collected log data as a feature which is analyzed by the artificial intelligence-based prediction model to be described below to deduce the prediction information about the occurrence of the mood episode of the user. Such feature information may be determined in advance in consideration of a type of a specific mood episode to be predicted using the artificial-intelligence prediction model and a mood disorder type of the target user 1.

To be more specific, when the target user 1 corresponds to a major depressive disorder (MDD) based on user information including disease information of the target user 1, the predicting apparatus 100 disclosed in the present disclosure may operate to calculate prediction information including information about an occurrence possibility of the major depressive episode (MDE) using an artificial intelligence-based analysis model. As another example, when the target user 1 corresponds to Bipolar I disorder, the predicting apparatus 100 may operate to calculate prediction information including information about the occurrence possibility of each of the major depressive episode (MDE), the manic episode (ME), and the hypomanic episode (HME) using an artificial intelligence-based analysis model. As still another example, when the target user 1 corresponds to Bipolar II disorder, the predicting apparatus 100 may operate to calculate prediction information including information about the occurrence possibility of each of the major depressive episode (MDE) and the hypomanic episode (HME).

With regard to this, according to the exemplary embodiment of the present disclosure, the predicting apparatus 100 individually trains (builds) a plurality of artificial-intelligence based predictions models including a first prediction model to output information about an occurrence possibility of the major depressive episode (MDE) as prediction information, a second prediction model to output information about an occurrence possibility of the manic episode (ME) as prediction information, and a third prediction model to output information about an occurrence possibility of the hypomanic episode (HME) as prediction information. In addition, the predicting apparatus determines a type of a mood episode required to be predicted in accordance with the target user 1 based on the disease information of the target user 1, and may operate to predict a user-customized mood episode occurrence (relapse) by utilizing an appropriate prediction model among the plurality of prediction models according to the determined type of the mood episode.

To be more specific, according to the exemplary embodiment of the present disclosure, when the disease information of the target user 1 corresponds to the major depressive disorder (MDD), the predicting apparatus 100 deduces information about the occurrence possibility of the major depressive episode (MDE) as prediction information by means of analysis using the first prediction model described above. When the disease information of the target user 1 corresponds to the bipolar I disorder, the predicting apparatus 100 deduces information about the occurrence possibilities of the major depressive episode (MDE), the manic episode (ME), and the hypomanic episode (HME) as prediction information by means of analysis using the first prediction model, the second prediction model, and the third prediction model. Further, When the disease information of the target user 1 corresponds to the bipolar II disorder, the predicting apparatus 100 may deduce information about the occurrence possibilities of the major depressive episode (MDE) and the hypomanic episode (HME) as prediction information by means of analysis using the first prediction model and the third prediction model.

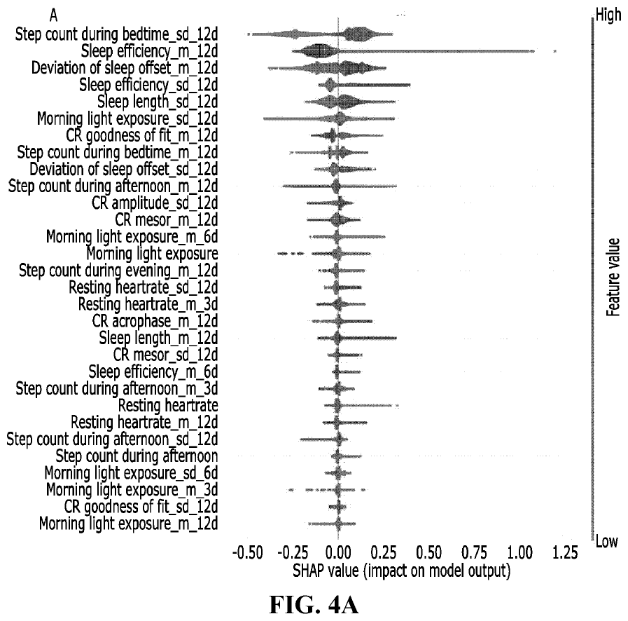

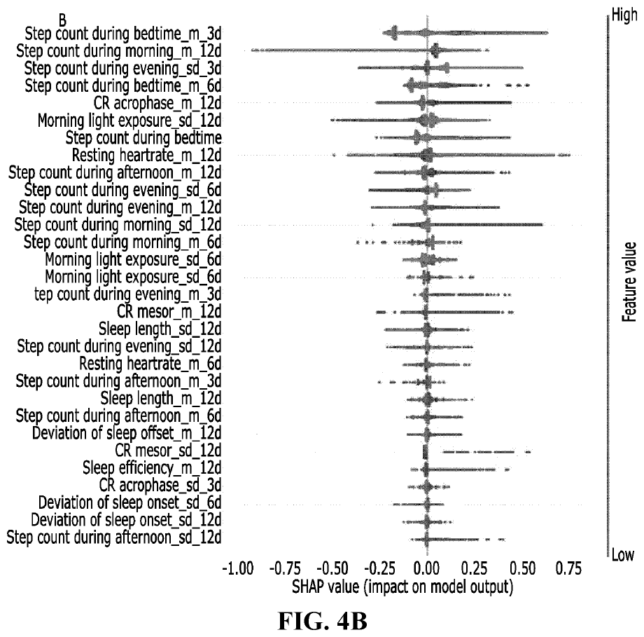

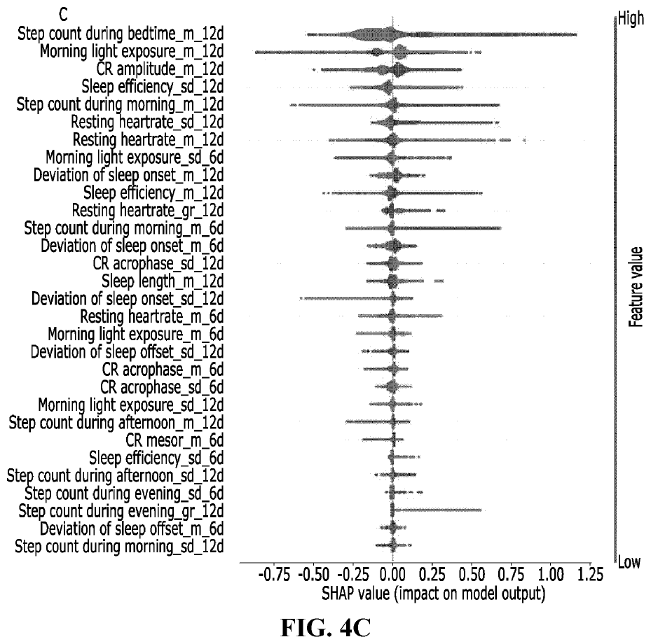

In the meantime, main feature information which is mainly considered by the plurality of artificial-intelligence based prediction models to deduce the prediction information, among the feature information deduced from the log data may be determined in different forms depending on the type of the prediction model and the main feature information according to the type of the prediction information will be described in more detail with reference to FIGS. 4A to 4C.

In summary, the predicting apparatus 100 may acquire user information including disease information about the mood disorder type of the target user 1 and deduce the prediction information using the target prediction model which is an artificial intelligence model determined to correspond to the disease information of the target user 1, among the plurality of previously trained prediction models, to be customized for the target user 1.

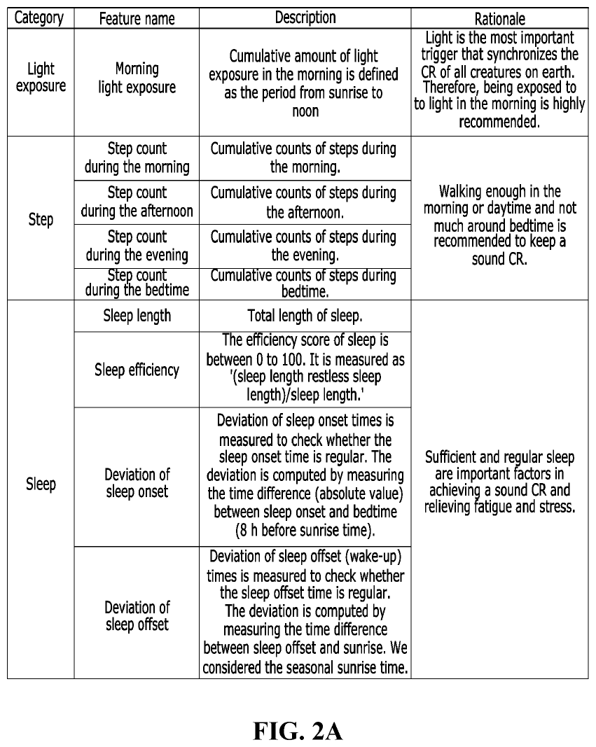

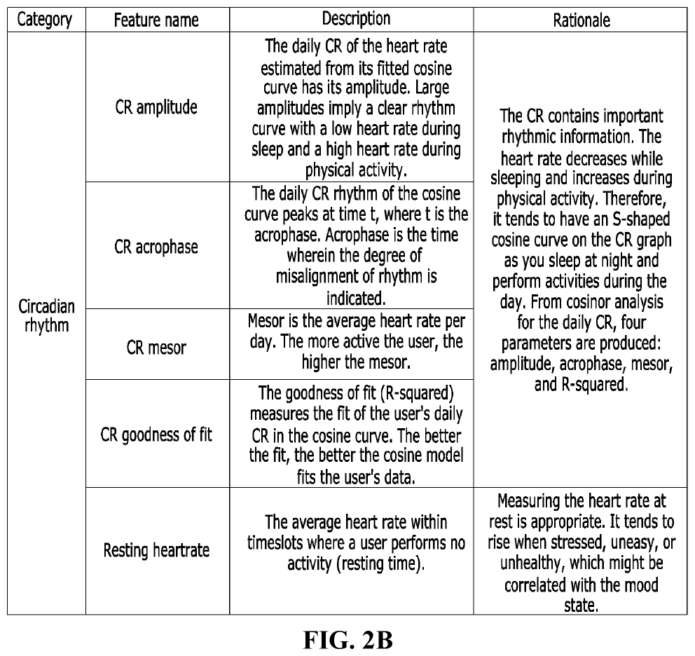

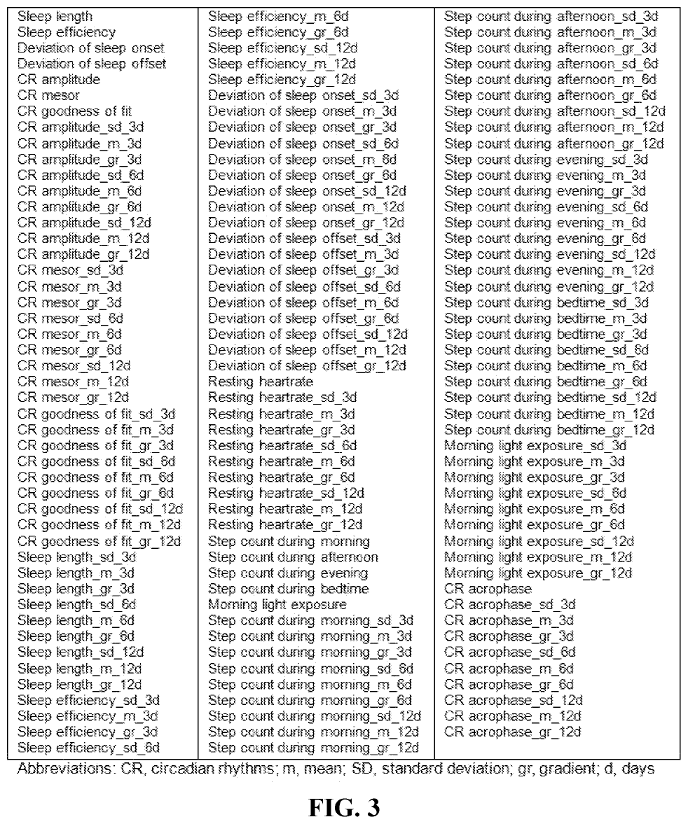

Hereinafter, a type of the feature information deduced from the log data will be described in more detail with reference to FIGS. 2A to 3.

FIGS. 2A and 2B are views for explaining a category of feature information deduced from log data collected for a user.

Referring to FIGS. 2A and 2B, the feature information deduced from the log data collected for the target user 1 may be classified into categories including light exposure information (light exposure), step information (step), sleep information (sleep), and heart rate information (HR) to be collected.

Further, according to the exemplary embodiment of the present disclosure, the predicting apparatus 100 may collect the light exposure information and the step information separately for each of a plurality of timelines which is divided in advance. To be more specific, the plurality of time slots may be obtained by dividing one day into a daytime and a bedtime or dividing one day into three time slots by 8 hours based on a sunrise time or a sunset time, but it is not limited thereto. When one day is divided into two time slots, the plurality of time slots may be equally divided by 12 hours to be the daytime and the bedtime, but it is not limited thereto. Therefore, the plurality of time slots may be unequally divided in accordance with a region from which the feature information is collected, the season, the date, and the climate (for example, the bedtime is from eight hours before the sunrise time to the sunrise time and the daytime is the remaining time slot (16 hours)).

The feature information which is collected for every category will be described in more detail. The light exposure information category may include cumulative light exposure information within a period from a sunrise to the noon on the corresponding day. With regard to this, exposure to the light acts as a major trigger to synchronize circadian rhythms of all creatures on earth so that a degree of the light exposure in the morning may mainly affect the mood episode occurrence of the target user 1.

Next, the step information category may include cumulative step count information for every time slot, such as morning, afternoon, evening, and nighttime (bedtime). With regard to this, in order to keep a circadian rhythm in a healthy state, it is necessary to walk enough in an active time slot such as morning or daytime and to less active at the nighttime.

Next, the sleep information category may include subcategories including a sleep length, a sleep efficiency, a deviation of sleep onset, and a deviation of sleep offset. Specifically, the sleep length may refer to a time from a timing that the sleep starts to a timing that the sleep ends on the corresponding day. According to the exemplary embodiment of the present disclosure, the timing that the sleep of the target user 1 starts or the timing that the sleep of the target user 1 ends may be determined by a user input (for example, a user input indicating to start the sleep before the sleep is applied or a user input indicating to end the sleep and wake up after waking-up is applied) which is directly applied to at least one of the user terminal 220 and the wearable device 210 by the target user 1. Alternatively, the timing that the sleep of the target user 1 starts or the timing that the sleep of the user 1 ends may be deduced by considering the remaining feature information, such as the step information or the light exposure information of the target user 1 acquired from at least one of the user terminal 220 and the wearable device 210.

Here, the sleep length is feature information corresponding to the sleep length as the entire sleep time and to be more specific, according to the implemented example of the present disclosure, may also be calculated by sub-divided into a time (restless sleep length) which is evaluated that the target user 1 spent uneasy sleep state. Further, for example, the sleep efficiency may be information collected from the wearable device 210 having a function of evaluating a sleep state or toss and turns of the target user 1 during sleeping when the target user 1 falls asleep while wearing the wearable device 210.

Further, the deviation of sleep onset is feature information indicating whether the target user 1 regularly starts to sleep and may be a deviation between a reference timing (for example, eight hours before the sunrise time) and the sleep onset time of the target user 1. Further, the deviation of sleep offset is feature information indicating whether the target user 1 regularly ends the sleep and may be a deviation between a reference timing (for example, the sunrise time) and the sleep offset time of the target user 1.

Next, the circadian rhythm category may include subcategories of a CR amplitude, a CR acrophase, a CR mesor, a CR goodness of fit, and a resting heartrate in a heart rate fitting curve deduced based on a number of heart rates of the target user 1 per unit time.

With regard to this, the feature information of the circadian rhythm category includes main rhythm information related to a heart rate HR of the user and the heart rate of the user may show a tendency to drop during the sleep and increase during the activity. Therefore, the heart rate fitting curve of the target user 1 who sleeps during the bedtime and does many activities during the daytime may show an ideal cosine curve similar to S.

Specifically, the CR amplitude in the heart rate fitting curve refers to an amplitude in a heart rate fitting curve having a cosine curve shape. The larger the amplitude, the clearer the heart rate rhythm. With regard to this, when the amplitude is relatively large, the user 1 may be in active and when the amplitude is relatively small, the target user 1 may be in sleep.

Further, the CR acrophase refers to how much the heart rate rhythm is misaligned, which quantifies the phenomenon that the biological rhythm (for example, the heart rate fitting curve) is pushed or pulled on the time axis. With regard to this, it is proven that a phenomenon that the biological rhythm is pushed or pulled and the occurrence of the mental disorder such as manic or depression are correlated.

Further, the CR goodness of fit indicates how well the heart rate fitting curve faithfully represents original sample data and may have a value between 0 and 1. As the CR goodness of fit is closer to 1, the cosine curve is more surely fitted. In contrast, as the coefficient is closer to 0, it means that the cosine curve is not satisfactorily fitted. That is, as the heart rate fitting curve is well fitted to the shape of the cosine curve, a value of R-square R² may be increased.

Further, the resting heartrate may refer to an average heart rate in a time slot when there is no activity of the target user 1. Specifically, the resting heartrate may increase when the target user 1 is stressed or in an anxious state, so that the resting heartrate may be utilized as the feature information related to the occurrence possibility of the mood episode of the target user 1.

FIG. 3 is a view for explaining a detailed type of feature information deduced from log data collected for a user.

Referring to FIG. 3, the predicting apparatus 100 accumulates the log data during a predetermined collection period and applies a predetermined statistical analysis method to the accumulated log data to deduce the feature information. In this case, the length of the collection periods is variably applied and various types of a statistical value calculating method to deduce a plurality of feature information (for example, 140 features illustrated in the table of FIG. 3) corresponding to the categories described above with reference to FIGS. 2A and 2B.

For example, referring to FIG. 3, the collection (accumulation) period of the log data may be divided into three days (3d), six days (6d), and 12 days (12d) and the statistical value calculating method for the accumulated log data may include a mean m, a standard deviation sd, and a gradient g.

Hereinafter, a type of main feature information which is determined so as to corresponding to each of the plurality of artificial-intelligence based prediction information which is individually built in consideration of a type of mood disorder and a type of a mood episode to be predicted, among the plurality of feature information, will be described for every prediction model and a process of building (training) the artificial-intelligence based prediction model will be described with reference to FIGS. 4A to 4C.

The predicting apparatus 100 may collect log data related to a circadian rhythm of a target user 1 from at least one of the user terminal 220 of the target user 1 and a wearable device 210 which is attached to or worn on a body of the target user 1. For example, a plurality of users selected to collect log data which is learning data to train the artificial intelligence-based prediction model may be selected as users corresponding to a mood disorder according to disease types of the major depressive disorder, the bipolar I disorder, and the bipolar II disorder.

Further, the predicting apparatus 100 may prepare answer data connected to the mood episode occurrence of each of the plurality of selected users. For example, the predicting apparatus 100 provides predetermined questionnaire items with contents associated with a mood episode occurrence to the user terminal 220 of each of the plurality of users at every predetermined examination period and may acquire an input applied to each user terminal 220 in response to the predetermined questionnaire items as answer data.

To be more specific, the predicting apparatus 100 may transmit a signal requesting to perform the ecological momentary assessment to a dedicated application (eMoodChart) which has been installed in advance in the user terminal 220 of each of the plurality of users. The questionnaire items for this assessment may be divided into levels of very low (−3), moderately low (−2), slightly low (−1), normal (0), slightly high (1), moderately high (2), and very high (3). The user terminal 220 may supply the answer data about each user's mood state and energy level to the predicting apparatus 100 using the questionnaire items.

Further, the predicting apparatus 100 may select main feature information associated with the occurrence of a specific type of mood episode, among a plurality of feature information which can be extracted from the log data based on the collected log data and the answer data. For example, the predicting apparatus 100 may select the main feature information associated with the occurrence of the major depressive episode (MED) based on the log data collected for the user corresponding to the major depressive disorder, the bipolar I disorder, or the bipolar II disorder and the answer data supplied by the user. As another example, the predicting apparatus 100 may select the main feature information associated with the occurrence of the manic episode (ME) based on the log data collected for a user corresponding to the bipolar I disorder and the answer data supplied by the corresponding user. As still another example, the predicting apparatus 100 may select the main feature information associated with the occurrence of the hypomanic episode (HME) based on the log data collected for a user corresponding to the bipolar I disorder or the bipolar II disorder and the answer data supplied by the corresponding user.

With regard to this, FIGS. 4A to 4C are graphs visualizing and illustrating main feature information corresponding to each of a plurality of prediction models for predicting the occurrence probability of a major depressive episode, a manic episode, and a hypomanic episode, respectively, and the degree of influence of each of the main feature information on the output of the prediction model.

Specifically, FIG. 4A illustrates the top 30 feature information which has the most important effect on the output of the first prediction model which outputs information about the occurrence possibility of the major depressive episode (MDE), among all the 140 feature information as prediction information, as main feature information associated with the first prediction model. Specifically, FIG. 4B illustrates the top 30 feature information which has the most important effect on the output of the second prediction model which outputs information about the occurrence possibility of the manic episode (ME), among all the 140 feature information as prediction information, as main feature information associated with the second prediction model. Specifically, FIG. 4C illustrates the top 30 feature information which has the most important effect on the output of the third prediction model which outputs information about the occurrence possibility of the hypomanic episode (HME), among all the 140 feature information as prediction information, as main feature information associated with the third prediction model.

For reference, the predicting apparatus 100 may perform shaplely additive explanations (SHAP) based analysis to determine main feature information which has a main important effect on the output value of each prediction model, among all the feature information, and visualize the degree of influence of the main feature information.

Further, in FIGS. 4A to 4C, each dot corresponds to a sample per day and red represents a higher feature value, and blue represents a relatively lower feature value. Further, the positive SHAP value (a SHAP value on a horizontal axis) indicates that the occurrence possibility of the corresponding episode is higher and specifically, and a red dot having the positive SHAP value represents that a tendency that the high feature value increases the occurrence possibility of the episode in the corresponding prediction model type is deduced.

Further, when the log data of the target user 1 is input, the predicting apparatus 100 may build the artificial intelligence-based prediction model which outputs the prediction information about the mood episode occurrence of the target user 1 using the main feature information determined by an episode type or a disease type.

Further, with regard to the prediction model implemented by the machine learning, according to the exemplary embodiment of the present disclosure, the prediction model may be trained based on a supervised learning based random forest algorithm, but it is not limited thereto and in the present disclosure, various machine learning algorithm models which have been known in the related art or will be developed in the future may be applied.

In summary, a type of the main feature information which is input to the target prediction model and a weight of each main feature information may be determined in consideration of the disease information of the target user 1.

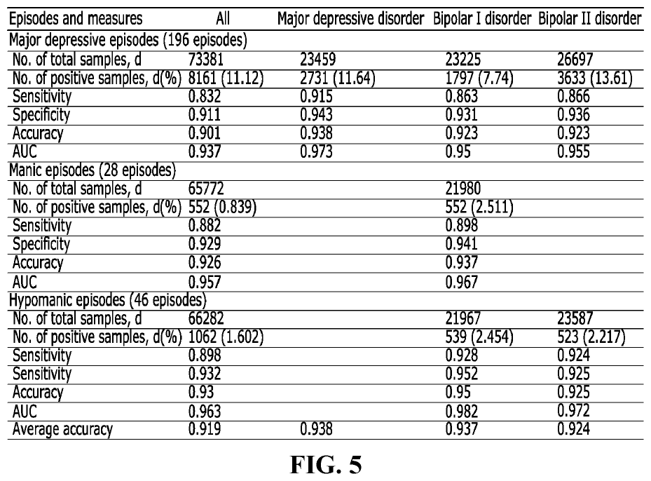

FIG. 5 is a table illustrating a prediction performance of each artificial intelligence-based prediction model of an apparatus for predicting an occurrence of a mood episode using a digital phenotype according to an exemplary embodiment of the present disclosure, according to a disease type.

Referring to FIG. 5, according to one experimental example associated with a mood episode occurrence prediction technique using a digital phenotype according to an exemplary embodiment of the present disclosure, it may be confirmed that average prediction accuracies for all mood episode types during a predetermined analysis period (for example, three days) were 91.9%, 93.8%, 93.7%, and 92.4% for all the participants, a participant group corresponding to the major depressive disorder (MDD), a participant group corresponding to the bipolar I disorder (BDI), and a participant group corresponding to the bipolar II disorder (BDII), respectively. Further, it may be confirmed that high prediction accuracies of each of the major depressive episode (MDE), the manic episode (ME), and the hypomanic episode (HME) for all the participants of 90.1%, 92.6%, and 93% were obtained.

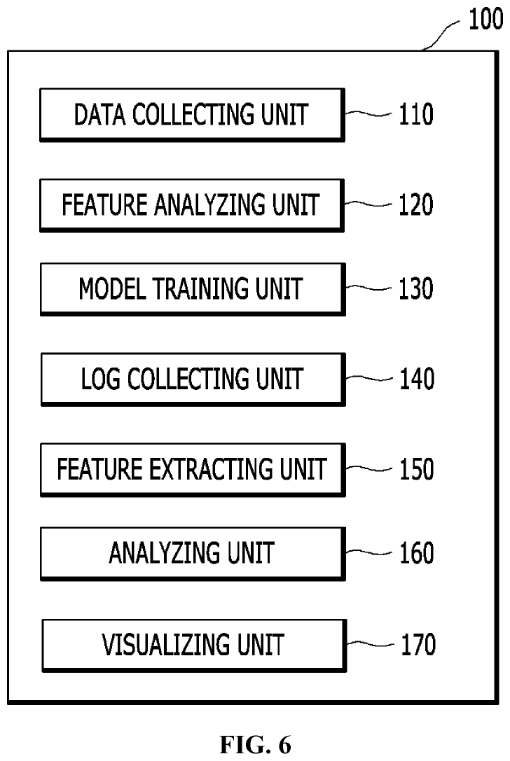

FIG. 6 is a schematic diagram of an apparatus for predicting an occurrence of a mood episode using a digital phenotype according to an exemplary embodiment of the present disclosure.

Referring to FIG. 6, the predicting apparatus 100 may include a data collecting unit 110, a feature analysis unit 120, a model training unit 130, a log collecting unit 140, a feature extracting unit 150, an analysis unit 160, and a visualizing unit 170.

The data collecting unit 110 may collect log data related to a circadian rhythm of each of a plurality of users collected from at least one of the user terminal 220 of each of a plurality of users and a wearable device 210 which is attached to or worn on a body of each of a plurality of users.

Further, the data collecting unit 110 may prepare answer data connected to the mood episode occurrence of each of the plurality of users.

The feature analysis unit 120 may select main feature information associated with the mood episode occurrence, among a plurality of feature information extracted from the log data based on the collected log data and the answer data.

When the log data of the target user 1 is input, the model training unit 130 may build the artificial intelligence-based prediction model which outputs the prediction information about the mood episode occurrence of the target user 1.

To be more specific, the log data and the answer data may be classified into a plurality of groups according to a mood disorder type of each of the plurality of users and the model training unit 130 may train a plurality of prediction modes corresponding to each mood disorder type using the classified log data and answer data.

The log collecting unit 140 may acquire log data related to a circadian rhythm of the target user 1 from at least one of the user terminal 220 of the target user 1 and a wearable device 210 which is attached to or worn on a body of the target user 1. Further, the log collecting unit 140 may acquire user information including disease information about the mood disorder type of the target user 1.

The feature extracting unit 150 may extract predetermined main feature information from the log data. Specifically, the feature extracting unit 150 may deduce main feature information which is set in advance so as to correspond to the disease information of the target user 1 from the log data.

The analysis unit 160 may deduce prediction information about the mood episode occurrence of the target user 1 by inputting the extracted main feature information to a previously trained artificial intelligence-based prediction model. Specifically, the analysis unit 160 may calculate information about the occurrence possibility of at least one of the major depressive episode (MDE), the manic episode (ME), and the hypomanic episode (HME) of the target user 1 within a predetermined analysis period as prediction information.

According to the exemplary embodiment of the present disclosure, the analysis unit 160 may deduce prediction information using a target prediction model determined so as to correspond to the disease information of the target user 1, among a plurality of previously trained prediction models.

The visualizing unit 170 evaluates the degree of influence on the calculated prediction information in which each main feature influenced and may visualize and display the evaluated degree of influence. For example, the visualizing unit 170 may evaluate the degree of influence of each main feature information through shaplely additive explanations (SHAP) based analysis.

Hereinafter, an operation flow of the present disclosure will be described in brief based on the above detailed description.

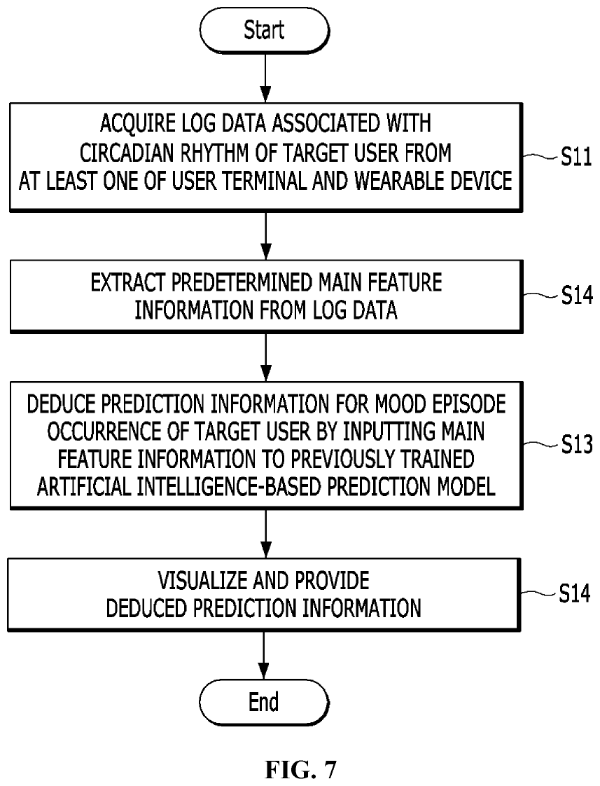

FIG. 7 is an operation flowchart of a method for predicting an occurrence of a mood episode using a digital phenotype according to an exemplary embodiment of the present disclosure.

A method for predicting an occurrence of a mood episode using a digital phenotype illustrated in FIG. 7 may be performed by the predicting apparatus 100 described above. Therefore, even though some contents are omitted below, the contents which have been described for the predicting apparatus 100 may be applied to the description for the method for predicting the occurrence of a mood episode using a digital phenotype in the same way.

Referring to FIG. 7, in step S11, the log collecting unit 140 may acquire log data related to a circadian rhythm of the target user 1 from at least one of the user terminal 220 of the target user 1 and a wearable device 210 which is attached to or worn on a body of the target user 1.

Further, in step S11, the log collecting unit 140 may acquire user information including disease information about the mood disorder type of the target user 1.

Next, in step S12, the feature extracting unit 150 may extract predetermined main feature information from the log data.

According to the exemplary embodiment of the present disclosure, in step S12, the feature extracting unit 150 may deduce main feature information which is set in advance so as to correspond to the disease information of the target user 1 from the log data.

Next, in step S13, the analysis unit 160 may deduce prediction information about the mood episode occurrence of the target user 1 by inputting the extracted main feature information to a previously trained artificial intelligence-based prediction model.

Specifically, in step S13, the analysis unit 160 may calculate information about the occurrence possibility of at least one of the major depressive episode (MDE), the manic episode (ME), and the hypomanic episode (HME) of the target user 1 within a predetermined analysis period as prediction information.

According to the exemplary embodiment of the present disclosure, in step S13, the analysis unit 160 may deduce prediction information using a target prediction model determined so as to correspond to the disease information of the target user 1, among a plurality of previously trained prediction models.

Next, in step S14, the visualizing unit 170 evaluates a degree of influence on the calculated prediction information in which each main feature is influenced and may visualize and display the evaluated degree of influence. For example, in step S14, the visualizing unit 170 may evaluate the influence of the main feature information through shaplely additive explanations (SHAP) based analysis.

In the above-description, steps S11 to S14 may be further divided into additional steps or combined as smaller steps depending on an implementation example of the present disclosure. Further, some steps may also be omitted if necessary and the order of steps may also be changed.

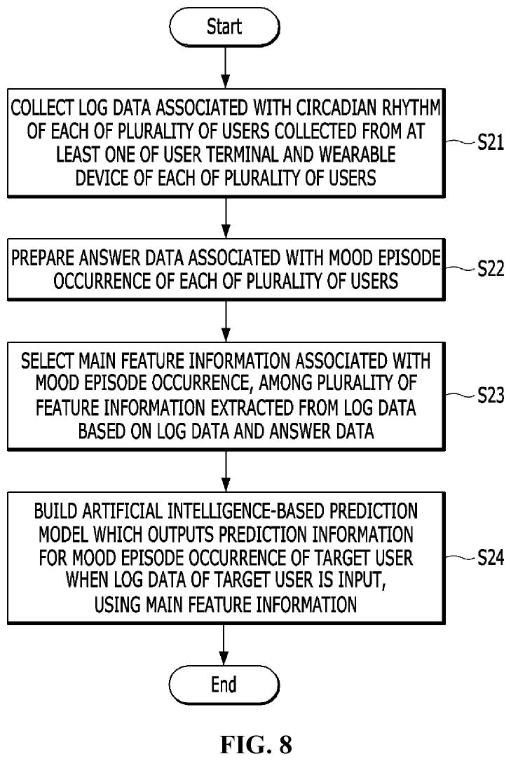

FIG. 8 is an operation flowchart of a training method of an artificial intelligence-based prediction model for predicting a relapse of a mood episode using a digital phenotype according to an exemplary embodiment of the present disclosure.

A training method of an artificial intelligence-based prediction model for predicting a relapse of a mood episode using a digital phenotype illustrated in FIG. 8 may be performed by the predicting apparatus 100 described above. Therefore, even though some contents are omitted, the contents which have been described for the predicting apparatus 100 may be applied to the description of FIG. 8 in the same way.

Referring to FIG. 8, in step S21, the data collecting unit 110 may collect log data related to a circadian rhythm of each of a plurality of users from at least one of the user terminal 220 of each of a plurality of users and a wearable device 210 which is attached to or worn on a body of each of a plurality of users.

Next, in step S22, the data collecting unit 110 may prepare answer data corresponding to the mood episode occurrence of each of the plurality of users.

Next, in step S23, the feature analysis unit 120 may select main feature information associated with the mood episode occurrence, among a plurality of feature information extracted from the log data based on the collected log data through step S21 and the acquired answer data through step S22.

Next, in step S24, when the log data of the target user 1 is input, the model learning unit 130 may build the artificial intelligence-based prediction model which outputs the prediction information about the mood episode occurrence of the target user 1.

To be more specific, the log data and the answer data may be classified into a plurality of groups according to a mood disorder type of each of the plurality of users and in step S24, the model training unit 130 may train a plurality of prediction models corresponding to each mood disorder type using the classified log data and answer data.

In the above-description, steps S21 to S24 may be further divided into additional steps or combined as smaller steps depending on an implementation example of the present disclosure. Further, some steps may also be omitted if necessary and the order of steps may also be changed.

The method for predicting an occurrence of a mood episode using a digital phenotype and the training method of an artificial intelligence-based prediction model for predicting a mood episode relapse using a digital phenotype according to the exemplary embodiment of the present disclosure are implemented in the form of a program instruction to be performed by various computer means to be recorded in a computer readable medium. The computer readable medium may include solely a program command, a data file, and a data structure or a combination thereof. The program instruction recorded in the medium may be specifically designed or constructed for the present disclosure or known to those skilled in the art of a computer software to be used. Examples of the computer readable recording medium include a magnetic media such as a hard disk, a floppy disk, or a magnetic tape, an optical media such as a CD-ROM or a DVD, a magneto-optical media such as a floptical disk, and a hardware device which is specifically configured to store and execute the program command such as a ROM, a RAM, and a flash memory. Examples of the program command include not only a machine language code which is created by a compiler but also a high level language code which may be executed by a computer using an interpreter. The hardware device may operate as one or more software modules in order to perform the operation of the present disclosure, and vice versa.

Further, the method for predicting an occurrence of a mood episode using a digital phenotype and the training method of an artificial intelligence-based prediction model for predicting a relapse of a mood episode using a digital phenotype described above may also be implemented as a computer program or an application which is recorded in a recording media to be executed by a computer.

The above description of the present disclosure is illustrative only and it is understood by those skilled in the art that the present disclosure may be easily modified to another specific type without changing the technical spirit of an essential feature of the present disclosure.

Thus, it is to be appreciated that the embodiments described above are intended to be illustrative in every sense, and not restrictive. For example, each component which is described as a singular form may also be divided to be implemented and similarly, components which are described as a divided form may be combined to be implemented.

The scope of the present disclosure is represented by the claims to be described below rather than the detailed description, and it is to be interpreted that the meaning and scope of the claims and all the changes or modified forms derived from the equivalents thereof come within the scope of the present disclosure.

What is claimed is:

1. A method for predicting an occurrence of a mood episode using a digital phenotype, comprising:
acquiring log data associated with a circadian rhythm of a target user from at least one of a user terminal of the target user and a wearable device which is attached to or worn on a body of the target user;
extracting predetermined main feature information from the log data; and
deducing prediction information about the occurrence of the mood episode of the target user by inputting the extracted main feature information to a previously trained artificial intelligence-based prediction model.

2. The method for predicting according to claim 1, wherein in the deducing of prediction information, information of an occurrence possibility of at least one of a major depressive episode, a manic episode, and a hypomanic episode of the target user is calculated as the prediction information within a predetermined analysis period.

3. The method for predicting according to claim 1, further comprising:
acquiring user information including disease information about a mood disorder type of the target user,
wherein in the deducing of prediction information, the prediction information is deduced using a target prediction model which is determined so as to correspond to the disease information, among a plurality of previously trained prediction models.

4. The method for predicting according to claim 3, wherein the mood disorder type includes a major depressive disorder, a bipolar I disorder, and a bipolar II disorder.

5. The method for predicting according to claim 3, wherein a type of the main feature information which is input to the target prediction model and a weight of each of the main feature information are determined in consideration of the disease information.

6. The method for predicting according to claim 1, wherein the main feature information includes feature information corresponding to each of a plurality of categories including heart rate information, light exposure information, sleep information, and step information of the target user.

7. A training method of an artificial intelligence-based prediction model for predicting a relapse of a mood episode using a digital phenotype, comprising:
collecting log data associated with a circadian rhythm of each of a plurality of users collected from at least one of a user terminal of each of the plurality of users and a wearable device which is attached to or worn on a body of each of the plurality of users;
preparing answer data associated with a mood episode occurrence of each of the plurality of users;
selecting main feature information associated with the mood episode occurrence, among a plurality of feature information extracted from the log data based on the log data and the answer data; and
building an artificial intelligence-based prediction model which outputs prediction information about mood episode occurrence of the target user using the main feature information when the log data of the target user is input.

8. The training method according to claim 7, wherein the log data and the answer data are classified into a plurality of groups depending on a mood disorder type of each of the plurality of users, and in the building of a prediction model, a plurality of prediction models corresponding to each of the mood disorder types is trained using the log data and the answer data which are classified.

9. The training method according to claim 8, wherein the mood disorder type includes a major depressive disorder, a bipolar I disorder, and a bipolar II disorder.

10. The training method according to claim 7, wherein the prediction information includes information about an occurrence possibility of at least one of a major depressive episode, a manic episode, and a hypomanic episode of the target user within a predetermined analysis period.

11. An apparatus for predicting an occurrence of a mood episode using a digital phenotype, comprising:
a log collecting unit configured to acquire log data associated with a circadian rhythm of a target user from at least one of a user terminal of the target user and a wearable device which is attached to or worn on a body of the target user;
a feature extracting unit configured to extract predetermined main feature information from the log data; and
an analyzing unit configured to deduce prediction information about the occurrence of the mood episode of the target user by inputting the main feature information to a previously trained artificial intelligence-based prediction model.

12. The apparatus for predicting according to claim 11, wherein the analyzing unit calculates information of an occurrence possibility of at least one of a major depressive episode, a manic episode, and a hypomanic episode of the target user as the prediction information within a predetermined analysis period.

13. The apparatus for predicting according to claim 11, wherein the log collecting unit further acquires user information including disease information about a mood disorder type of the target user, and the analyzing unit deduces the prediction information using a target prediction model which is determined so as to correspond to the disease information, among a plurality of previously trained prediction models.

14. A training apparatus for an artificial intelligence-based prediction model for predicting a relapse of a mood episode using a digital phenotype, comprising:
a data collecting unit configured to collect log data associated with a circadian rhythm of each of a plurality of users collected from at least one of a user terminal of each of the plurality of users and a wearable device which is attached to or worn on a body of each of the plurality of users, and prepare answer data associated with occurrence of mood episode of each of the plurality of users;
a feature analyzing unit configured to select main feature information associated with the mood episode occurrence, among a plurality of feature information extracted from the log data based on the log data and the answer data; and
a model training unit configured to build an artificial intelligence-based prediction model which outputs prediction information about a mood episode occurrence of the target user using the main feature information when the log data of the target user is input.

15. The training apparatus according to claim 14, wherein the log data and the answer data are classified into a plurality of groups depending on a mood disorder type of each of the plurality of users, and the model training unit trains a plurality of prediction models corresponding to each of the mood disorder type using the classified log data and answer data.
